(19) United States
(12) Patent Application Publication
(10) Pub. No.: US 2010/0130949 A1
(43) Pub. Date: May 27, 2010

(54) Catheter drainage system

(76) Inventor: Maurice M. Garcia, San Francisco, CA (US)

Correspondence Address: QUINE INTELLECTUAL PROPERTY LAW GROUP, P.C., P O BOX 458, ALAMEDA, CA, 94501, US

(21) Appl. No.: 12/454,179

(22) Filed: May 12, 2009

Related U.S. Application Data
(60) Provisional application No. 61/211,873, filed on Apr. 3, 2009.
(60) Provisional application No. 61/127,930, filed on May 16, 2008.

(51) Int. Cl. A61M 27/00; A61M 1/00

(52) U.S. Cl. 604/326; 604/328

(57) ABSTRACT

This invention provides systems and methods to collect biological fluids from a catheterized or intubated animal. Drainage tubes are provided that reduce fluid back pressure by avoiding the formation of dependent loops. Low aspect ratio collection receptacles are provided that rest on a flat surface to improve fluid flows and/or minimize back-pressures exerted by collected fluids.

### CROSS-REFERENCE TO RELATED APPLICATIONS

This application claims priority to and benefit of a prior U.S. Provisional Application No. 61/211,873, A Passive, Continuous-flow, Gravity-dependent Body Fluid Drainage Bag System, Urometer, and Accessory Devices, by Maurice M. Garcia, Filed Apr. 3, 2009 and prior U.S. Provisional Application No. 61/127,930, A Passive, Continuous-flow, Gravity-dependent Body Fluid Drainage Bag System, Urometer, and Accessory Devices, by Maurice M. Garcia, Filed May 16, 2008.

### FIELD OF THE INVENTION

The present inventions are in the field of systems with bags or other receptacles to receive drainages, e.g., from biological sources, without a build up of back pressure. Included are methods of collecting biological fluids from an animal by catheterizing the animal and running the drain tube to a top center fill port inlet to a horizontally flattened bag. Such a bag has lower side wall tension and lower back pressure on filling than typical vertically hanging bags.

### BACKGROUND OF THE INVENTION

Foley catheter drainage bag kits possess a drainage tube (connecting a patient's catheter to a collection bag), which under conditions of “general use”, often assumes a dependant-curl position. When the dependant-most portion of the drainage tube fills with urine, an air-fluid lock develops, and subsequent drainage of urine from the patient into the drainage tubing encounters progressive backpressure from the air-fluid lock. This back-pressure opposes further drainage of fluid into the drainage tube, and the patient's bladder is forced to store newly produced urine. The drainage tube system ceases to drain the bladder until a sufficiently high bladder pressure is generated, sufficient to overcome the backpressure generated by the air-fluid lock.

A urinary drainage catheter, such as the Foley catheter, is a hollow, tubular device commonly used in the medical profession for insertion into a patient's bladder via the urethral tract to permit the drainage of urine. Use of a urinary catheter is often necessary for patients that are undergoing surgery, orthopedically incapacitated, incontinent, or incapable of voluntary urination. An unfortunate problem with catheterization, however, is the development of sepsis and/or urinary tract infections (UTIs) as a result of bacterial invasion in the bladder and urinary tract by various microorganisms. Urinary tract infection requires that the bacterial count surpass a particular threshold. The mere presence of a small number of bacteria are unlikely to cause a clinical infection, whereas proliferation beyond a particular threshold, depending on the bacteria, is much more likely to result in clinical infection. Sepsis is potentially lethal and most prevalent in the elderly, where urinary tract and bladder infections become systemic very easily, especially if hygiene is poor and hydration of tissue is deficient. A well-established risk factor for urinary tract infections is the presence of undrained volumes of urine within the bladder. Urine often contains proteins and other nutrients that aid bacterial growth and proliferation. For this reason, patients are encouraged to maintain their bladder as empty as possible with regular and complete voiding or self-catheterization (catheter is inserted by the patient into his/her bladder several times throughout the day, and removed immediately after the bladder is emptied).

The risk of sepsis increases with the employment of urinary drainage catheters, and particularly so when the catheter is left in-dwelling, as occurs more commonly in the hospital setting and/or in out-patients who are incapacitated. When the catheter is left indwelling, bacterial flora (e.g. from feces or local skin surfaces) can ascend along the outer walls and inner lumen of the catheter, into the bladder. When the bladder is maintained empty, bacteria that have ascended are less likely to grow and proliferate within the bladder. However, when the bladder contains undrained urine on a regular basis, any bacteria that ascend and make contact with urine are more likely to flourish and translocate to other areas of the urinary tract within the resultant contaminated urine.

In addition, residual urine in stasis around the retention balloon provides a culture medium at warm body temperatures that can facilitate the growth of bacteria both within the bladder and upon the catheter itself. Bacterial colonization results in their production of a proteinaceous material called “Biofilm”, which is accumulated upon the surfaces of the foreign body, and within which the bacteria reside. The biofilm that the bacteria secrete serves as a protective barrier. Consequently, bacteria are able to accumulate, multiply and become pathogenic in the bladder, eventually migrating into the kidneys and into the blood, resulting in sepsis. Because of this propensity to produce infection in the patient, medical practitioners often refuse to extend the use of catheters, despite their usefulness.

Urinary tract infections (UTI's) are the most common nosocomial infection, and greater than 90% of these are catheter related (Nicolle (2001) Infections in Medicine, 18: 153; Sedor and Mulholland (1999) Urol Clin North Am, 26: 821). Nosocomial UTI's are a source of increased morbidity, mortality, and increasing financial burden of healthcare systems worldwide, accounting for more than 1 million cases in U.S. hospitals annually (Foxman (2003) Dis Mon, 49: 53; Biering-Sorensen et al. (2001) Drugs, 61: 1275). Each episode of symptomatic nosocomial UTI adds nearly $700-1,500 dollars to the hospital bill (Saint (2000) Am J Infect Control, 28: 68), and an annual cost to the US healthcare system of nearly $451 million dollars (Jarvis (1996) Infect Control Hosp Epidemiol, 17: 552). Catheter-related bacteremia is estimated to cost nearly $2,900 per episode (Id.). Subpopulations at greatest risk for nosocomial catheter related UTI (the elderly, paraplegics, infants, pregnant women, diabetics, and patients with HIV/AIDS) (Id.).

The risk of UTI increases with increasing duration of catheterization. Recurrent infections lead to bacterial resistance to antibiotics. Long term catheterization has been associated with severe complications such as pyelonephritis (Warren (2001) Int J Antimicrob Agents, 17: 299; Huang et al. (2004) Infect Control Hosp Epidemiol, 25: 974), nephrolithiasis, epididymitis and prostatitis (Warren et al. (1994) J Am Geriatr Soc, 42: 1286). Bacteremia can occur when large static urine volumes and infection are combined with local urothelial trauma from chronic factors such as: catheter erosion, focal bladder wall ischemia due to persistent increased intraluminal pressures, and acute trauma from excessive catheter traction (Seiler and Stahelin (1988) Geriatrics, 43: 43). The discomfort associated with a distended bladder can caused unsupervised patients to pull their catheters out, resulting in urethral trauma/stricture, bleeding, and bacteremia.

Despite increasing numbers of patients with chronic indwelling Foley catheters, product innovation in this field has been limited to classes of material coatings designed to impede bacterial migration over the catheter and into the patient. Such new products have naturally focused on the urethral catheter component of the drainage system. For example, less reactive catheter materials such as silicone (Graiver et al. (1993) Biomaterials, 14: 465), low friction coatings such as Teflon, BN-74, and Hydrogel, and drug-eluting and silver impregnated surface coatings (Graiver et al. (1993) Biomaterials, 14: 465; Klarskov et al. (1986) Acta Obstet Gynecol Scand, 65: 295; Sabbuba et al. (2002) BJU Int, 89: 55; Gaonkar et al. (2003) Infect Control Hosp Epidemiol, 24: 506) were developed to decrease catheter-associated UTI's. These products have demonstrated inconclusive efficacy and unfavorable cost-effective value for even short-term prevention of urinary tract infections. No practical advances in product design have been made to improve long-term urinary catheter-related tract infection rates.

While bacteriostatic/bactericidal materials coatings active at the level of the catheter make intuitive sense to help prevent nosocomial UTI's, but such measures are ineffectual when persistent residual volumes of urine within the bladder serve as a medium for bacteria and source of infection.

Obstruction to bladder outflow has other deleterious effects aside from increased risk of infection. For example, a full and distended bladder is painful. In a disoriented patient, acute severe pain can sometimes cause the patient to violently withdraw the catheter from their body, resulting in severe injury to the urethra, bleeding, and risk of developing long-term sequellae, such as urethral stricture disease. When obstruction to drainage is unrelieved, spontaneous bladder rupture can occur, resulting in leakage of urine into inner cavities of the body, resulting in sepsis, electrolyte derangements, and possibly death. When bladder distension is chronic, normal bladder function declines and becomes increasingly irreversible. Long-term bladder dysfunction leads causes poor emptying, and elevated post-void residual volumes, and increased risk of infection.

Blockage is problem frequently reported by more than half of outpatients with chronic urinary catheters (Wilde (2003) J Adv Nurs, 43: 254; Kunin et al. (1987) J Urol, 138: 899). The literature suggests that the most common causes of catheter blockage include blood clots, sediment crystals and mucus within the catheter lumen (Getliffe (1994) J Adv Nurs, 20: 140). Catheter blockage accounts for many unscheduled office, evening and weekend visits, in addition to emergency room visits and visits by home nurses (Wilde (2002) Home Healthc Nurse, 20: 449). A study examining after-hours home care nursing calls notes that 22 of 25 patients reported catheter-related problems (Wilde (2003) J Adv Nurs, 43: 254).

One technology that addresses the problem of dependent curls in drainage tubes is described in U.S. patent application 2006/0271019 by Garcia and Stoller. A drainage collection system is configuration with a coil conformation imposed on the drainage tube segment. The downward-spiral conformation of the “absorbs” the redundant length of drainage tube, and maintains it the downward-spiral conformation such that no portion of the tubing is dependant, and all fluid drainage through the tubing is forced by gravity to migrate distally into the bag, and since no fluid collects within the tubing, fluid cannot create an obstructing air-fluid lock.

Garcia and Stoller also designed various devices to maintain the drainage tubing segment in a constantly downward-oriented direction, to preclude the formation of dependant curls, and such that all drainage inflow would be forced by gravity to migrate distally into the drainage bag. Examples of such devices include “support-arms”, which hold the drainage tube away from and below the level of the patient's bladder, again, maintaining the tube in a downward-pointing direction at all times. Other examples include a receptacle whose height is intermediate between the height of the patient's bladder, and the height of the drainage bag. The drainage bag is placed into the receptacle, and then the receptacle is maintained sufficiently away from the patient's bed such that all redundancy in the tubing is maintained in a downwardly oriented straight conformation. However, the drainage tube still must be attended and significant back-pressure can also originate in mechanical forces exerted on the collected fluid by the collection bag walls. Further, the weight of a semi-full bag within the receptacle can serve as a tether to the patient, and as such, be potentially dangerous. Use of such a receptacle can be awkward and difficult without the aid of another person to ensure that the receptacle is placed a sufficient distance from the patient.

In view of the above, a need exists for a drainage collection system that minimizes backpressures from the catheter through to the collection bag. It would be desirable to have collection system drainage tubes configured to further avoid the possibility of dependent curls. The present invention provides these and other features that will be apparent upon review of the following.

### SUMMARY OF THE INVENTION

Previous design solutions have approached the problem of eliminating dependant curls within the drainage tube segment, bearing the following key assumptions: 1) The drainage bag is always located in a vertical position (i.e. the generally circular-shaped bag is hung vertically nearby the patient); 2) the drainage tube enters the bag eccentrically (not in the center of the bag, but off-center, close to the highest point of the bag when the vertical bag; 3) the drainage tubing itself is always of a consistent caliber and set length. The present inventions combine collection system aspects stepping away from these old technologies.

The present inventions provide methods and devices for reducing the backpressure in a drainage tube between a catheter and a biological fluid collection bag. For example the drainage tube can drain into a flat collection bag on or near the floor. In this way, the drain tube tends to avoid loop configurations that result in trapping of air and fluid pockets. In addition, this innovation avoids the relatively high pressures encountered upstream of fluids collected in typical vertically hanging bags.

An exemplary device for collection of biological fluids can include a bag having an inner space between a top wall and a bottom wall, wherein the inner space is characterized by a width and/or a depth greater than the height. The device can have a drainage tube in fluid contact with the bag inner space through an inlet port located in the top wall so that the biological fluid flows from the drainage tube into the bag inner space. In use, the collected fluid has a greater breadth than height, even when the bag is full.

In preferred embodiments, the top wall and bottom wall are substantially planar and parallel when the inner space is empty of fluid. It is preferred that the top wall and/or the bottom wall consist of a flexible polymer sheet. In many embodiments, the walls are heat-sealed or sonically sealed together, e.g., so that the top wall and the bottom wall are in direct contact at a hermetically sealed peripheral edge of the top wall and/or the bottom wall. In many cases, the top wall and bottom wall are in direct contact with each other, e.g., at least at the entire peripheral edge of at least one of the walls. Optionally, there is a side wall of material interspersed between the top and bottom walls and bonded substantially perpendicular to the top and/or bottom wall. It is preferred that the width of the inner space is greater than the height of the inner space when the bag is full of the biological fluid, e.g., when the bag is resting with the bottom wall in contact with a horizontal surface. For example, it is preferred that the mid bag vertical cross-section be less than the mid bag horizontal cross-section, e.g., when the inlet port is uppermost. In a preferred embodiment, the bag is other than a bag comprising a mounting device for hanging the bag.

In optional embodiments, the inner space is vented or not vented to the external environment in use. Optionally, the inlet port is located in the top center of the top wall or is located between the top wall center and the peripheral edge, but is not in direct contact with the peripheral edge. Optionally, the drain tube or inlet port comprises a one way valve configured to allow fluid flow into the bag but not out of the bag.

In more preferred embodiments, the bag is configured so that there is less tension on the bag walls with the device resting on a horizontal surface with the inlet port uppermost, than the tension on the bag walls with the device resting on a horizontal surface with the inlet port positioned laterally.

In some embodiments, a fluid trapping loop is prevented in the drainage tube near floor level by provision of spacers mounted around the drainage tube so that the tube is held off the floor, and preferably held at a level above the inlet port.

Optional aspects can help keep the bag in place and provide a sanitary resting place for the collection bag. For example, the drainage collection system can further include a barrier under the bottom wall, thereby preventing contact of the bottom wall with a surface the device rests upon. Such barriers can include, e.g., a framework stand to hold collection bag off the floor, a basin to hold the bag, a pan, a bowl, a paper pad, and the like. The bag can be held in place on the floor by provision of a weight, suction cup or sticky surface mounted to an external surface of the bottom wall.

The drainage tube can have a length configured to avoid excess slack that can allow part of the tube to hang below adjacent parts of the tube forming location for capture of fluid in a dependent curl (dead leg, fluid trap). For example, the drainage tube can be configured have a telescoping length.

The present inventions include a urometer for precisely measuring the rate and/or volume of fluid drainage. The urometer can have a first chamber having a first volume and mounted within a second chamber in fluid contact with the second chamber through a conduit or port. The volume of the second chamber minus the volume of the first chamber can be at least 5-fold less, 10-fold less or 25-fold less than the volume of the first chamber. The urometer can have a drain tube in direct fluid contact with the second chamber. The urometer can have a second chamber external wall that is transparent and includes volumetric indication markings. The urometer can be configured so the second chamber empties into a first chamber that is a drainage bag having a top wall and bottom wall defining an inner space characterized by a width or a horizontal depth greater than a height.

The present inventions include methods of collecting a biological fluid from an animal. The methods can include the steps of catheterizing the animal with a catheter; providing a collection bag comprising an inner space defined between a top wall and a bottom wall, wherein the inner space is characterized by a width or a horizontal depth greater than a height; and wherein the drainage tube is in fluid contact with the bag inner space through an inlet port located within the top wall; and functionally connecting a drainage tube between the catheter and the collection bag to drain the biological fluid from the catheter into the bag inner space through the drainage tube. In preferred embodiments, the top wall is planar and substantially horizontal with the inlet port closer to a center of the top wall than to the peripheral edge of the top wall.

The collection bag can be mounted or resting at a location below a location where the catheter is catheterized into the animal. The collection bag can be placed on a horizontal surface with the bottom wall resting on the horizontal surface. The horizontal surface can be the floor of a room.

In another aspect, the inventions can include relatively solid low aspect ratio receptacles to receive fluids, e.g., such as thoracic fluids. The shorter receptacles are less easily tipped, receive large volumes, reduce the possibility of dependent loop formation in input lines, and provide optimum potential energy to drive fluids along the drainage tube. In many embodiments, the receptacle walls are not substantially flexible so that a relative low pressure (e.g., from a vacuum pump) can be applied within the system to enhance fluid drainage.

### DEFINITIONS

Unless otherwise defined herein or below in the remainder of the specification, all technical and scientific terms used herein have meanings commonly understood by those of ordinary skill in the art to which the present invention belongs.

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a component” can include a combination of two or more components; reference to “fluid” can include mixtures of fluids, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

As used herein, the directional terms refer to normal usage at locations on the surface of the earth. For example a top surface is above a bottom surface. Horizontal is perpendicular to the force of gravity and vertical is parallel to the force of gravity at in the local environment.

Substantially means largely or predominantly.

As used herein, the term “catheter” refers to a tubular medical device for insertion into canals, vessels, passageways, wound spaces or body cavities to permit drainage of biological fluids from an animal. The term can include a chest drainage tube.

A “biological fluid” refers to any one or more fluids produced by a biological organism. Such biological fluids include, but are not limited to urine, cerebral spinal fluid, blood or blood fractions, exudates, plasma, saliva or other oral fluid, gastrointestinal fluid, bile, pus, liquefied tissues, and the like.

An “aspect ratio” is the ratio between the cross-sectional height and cross-sectional width. A low aspect ratio is a ratio less than 1. In most embodiments of the present inventions, the collection receptacles of the systems have an aspect ratio, in use (e.g., with the inlet port above the internal volume), of 0.5 or less, 0.3 or less, 0.2 or less, 0.1 or less.

### BRIEF DESCRIPTION OF THE DRAWINGS

FIG. 1 is a frontal cross-sectional view schematic diagram of a drainage fluid collection system including a catheter, drainage tube and low aspect ratio collection bag.

FIGS. 2A and 2B are schematic diagrams of exemplary telescoping drainage tubes.

FIGS. 3A and 3B are schematic diagrams representing aspects of fluid drainage collection systems of the invention. FIG. 3A shows a catheter on a bed, draining to a flat collection bag on the floor through a drainage tube. FIG. 3B shows a substantially downward view of an exemplary low flat aspect ratio collection bag.

FIG. 4 is a schematic diagram of an exemplary system including a peripheral vertically standing urometer.

FIG. 5 is a schematic diagram of an exemplary collection system having a peripheral urometer having a side transfer tube.

FIG. 6 is a schematic diagram of a drainage collection system providing a gentle vacuum to enhance collection of certain fluids.

FIG. 7 is a schematic diagram of an old art three-bottle suction control and fluid collection system.

FIG. 8A is a schematic diagram showing how dependent loops in old art collection systems can change the pressures experienced in a patient's chest tube. FIG. 8B plots chest tube pressures versus dependent loop meniscus differential.

FIG. 9 shows how old art chambers and channels of a drainage tube collection unit (9A) can be incorporated into a low aspect ratio unit (9B) to provide lower back pressures in a drainage tube.

FIG. 10A shows a schematic diagram of an old art collection system. FIGS. 10B to 10E show schematic diagrams of alternate embodiments of low aspect ratio vacuum administration and fluid collection units.

FIG. 11 shows a schematic diagram of a fluid collection device having a suction control chamber mounted to a low aspect ratio water seal chamber.

FIG. 12 shows a schematic diagram of a collection device having a conical collar to receive tubing without formation of a dependent loop.

FIG. 13 shows a pressure relief valve incorporated into a drain tube.

### DETAILED DESCRIPTION

The devices and methods of the invention provide for collection of biological fluids drained from an animal. The devices include, e.g., catheters draining through drainage tubes to collection bags having a broad flat aspect ratio and resting flat on a horizontal surface. The devices can include, e.g., a urometer adjacent to, or surrounding, a much larger collection chamber for periodic measurement of biological fluid drainage rate and/or accumulated quantity. The methods of the invention include the steps of catheterizing an animal and draining a biological fluid into a collection device of the invention having a broad aspect ratio collection bag.

### Fluid Drainage Collection Systems

Fluid collection systems of the invention generally include, e.g., a catheter in fluid contact with a drainage tube that flows into the top of a flexible collection bag having a relatively flat horizontal aspect ratio in use. Such a design reduces the likelihood of experiencing back pressure at the catheter caused in traditional designs by elevation of the bags, by creation of low loop fluid traps in the drainage tube, and by hydraulic pressure in the collection bag due to side wall tension of traditional vertically hanging bags.

Bags in the inventive systems can have relatively low backpressures in drainage tubes, e.g., by resting the associated collection bag flatly on the floor. At this lowest collection level, the potential energy of siphoning is greatest and drainage is improved. Extending the collection bag to the floor can help straighten the drainage tube to reduce the formation of drooping loops (dependent curl or dead legs) that can collect fluid and restrict flow. With the bag on the floor, the drainage tube is unable to droop below the level of the collection bag. With the bag resting broadly on a horizontal surface, the depth of collected fluid is minimized, thus minimizing the fluid pressure at the bottom of the bag and the tension on the sides of the bag. With the collection bag broadly resting on the floor, side wall tension, and pressure exerted on collected fluid is reduced. None of these advantages are available in typical collection bags currently in use.

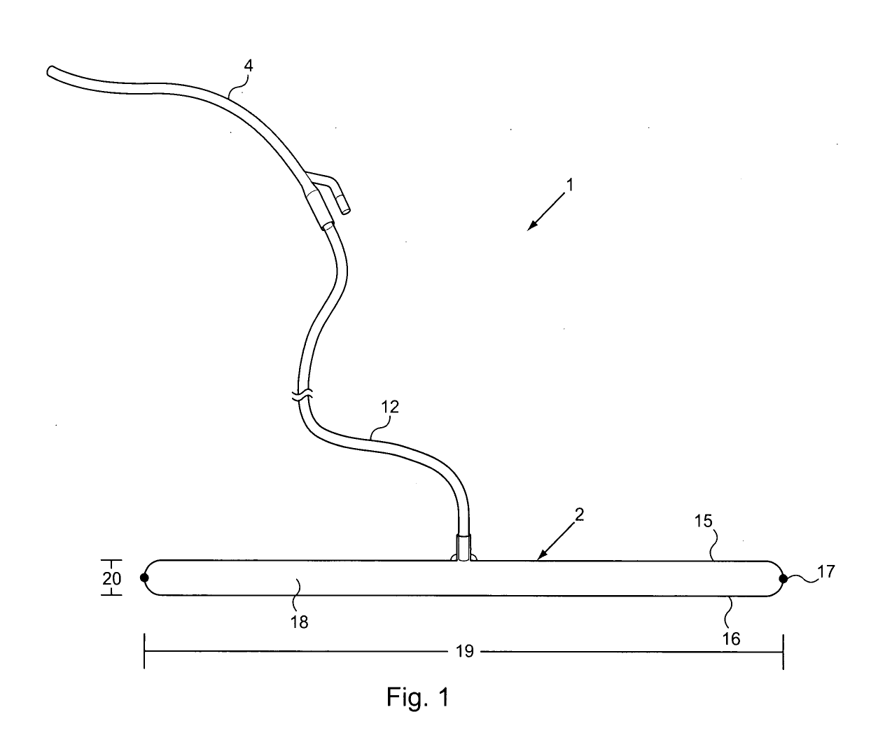

A typical fluid collection system 1 of the invention is shown in FIG. 1. The fluid collection system can include a catheter 4, connected in fluid contact with collection bag 2 through drainage tube 12. The collection bag can include a top wall 15 and a bottom wall 16 hermetically sealed together at peripheral edge 17. The top and bottom walls may contact, particularly when no fluid has been collected. However, typically the flexible wall sheets have an inner space 18 therebetween. The inner space has a width 19 greater that height 20 when the bag is empty (empty the space volume can approach zero) and typically also when the bag is full. The horizontal depth dimension, not shown, is also typically greater than the height dimension.

### Catheters

Catheters are typically tubular devices adapted to enter the body of an animal (e.g., a human medical patient) to contact a source of a fluid (e.g., a urine bladder or surgical wound space). Catheters provide a channel for fluids to flow from the source and out from the body. Once introduced into the body, catheters can be retained in position, e.g., by tissue resilience, adhesive tapes, or inflation of a chamber at the proximal end of the catheter, making it too large to exit through the body channel entered. The distal end of the catheter can have one or more connection means, such as a luer connection, for connection of the catheter to external conduits, such as drainage tubes.

Urine catheters allow drainage of urine from the bladder. Urine flowing from the catheter can flow through a drainage tube to a collection bag. Collected urine can be measured for adequate flow, analyzed for signs of infection or disease, or simply be discarded.

Wound drainage catheters are typically placed in the entry of a traumatic or surgical incision to allow drainage of wound fluids, such as blood and exudates that can accumulate causing pain, promote infection and slow the healing process. Collecting wound fluids to a sanitary collection vessel can, help keep bedding clean, prevent alteration of fluids for pathology analysis, and prevent spread of pathogens that may be associated with the fluid.

### Drainage Tubes

Drainage tubes are typically flexible transparent plastic tubes that direct drainage from a catheter to a collection bag. Drainage tubes of the invention can have one or more adaptations to prevent dependent curl collection of fluids between the catheter and collection bag. For example, the drainage tubes can extend vertically a greater distance than traditional tubes, the drainage tubes can have a length custom fit to the distance between the catheter and the collection bag, and/or spacers extending radially from the tubes can ensure the tube never droops below the level of the bag input port.

Because drainage collection systems of the invention can have the collection bag at the lowest possible position, the drainage tube can have a greater slope and greater potential energy for drainage. A typical old art drainage system may have an 18 inch drop. With the old bag hanging at a bed corner, to be out of the way of care providers, the drainage tube may have an average slope of 20 degrees, or less. The elevation change energy is low, and the near horizontal course allows gravity to pull the tube down into a dependent curl loop. The present systems often have a drop of 30 inches to a bag, which is out of the way on the floor, with an average slope near vertical. Such a system has the energy and low resistance to pull fluids away from the catheter and does not present an opportunity for gravity to pull the tube into a dependent curl.

Because the collection bags of the invention can be placed anywhere on the floor, as compared to the limited hanging bag opportunities, the distance between the catheter and bag is infinitely variable. That is, the bag can be moved any distance to customize the path length exactly fit the length of a given drainage tube (and patient size and patient posture), so that there is no opportunity for excess tubing to droop.

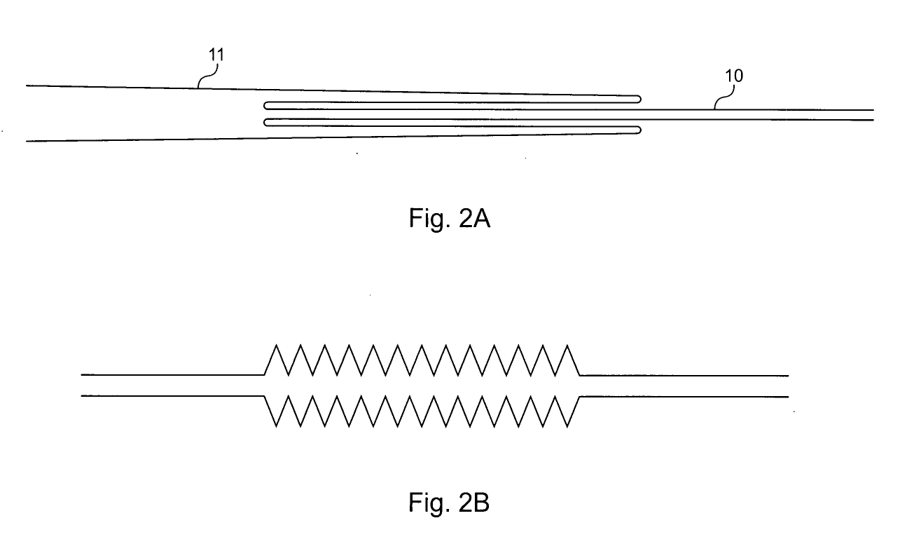

In another aspect of the invention, the length of drainage tubes themselves can be adjustable. For example, the drainage tube can be configured so that the length can be discretely adjusted (permanently, continuously or intermittently) so that excess slack does not exist in the tube between the catheter and collection bag. In one embodiment, shown in FIG. 2A, the drainage tube can have essentially an extended conical profile, e.g., tapering toward the bag or toward the catheter. Because the plastic tubes are somewhat resilient and, e.g., the cross-section is continuously changing, the narrow end 10 can be prolapsed into the wider end 11, thus shortening the overall length of the tube. In certain embodiments, the drainage tube can be provided with the proximal end (e.g., for attachment to the catheter) narrower with a section stuffed into the wider distal end (e.g., for connection to the bag inlet port). In use, the distal end can be attached to the bag on the floor and just enough of the proximal section pulled out of the distal section to reach the catheter in place, thus providing a drainage tube just the right length and without the possibility of a dependent curl. Optionally, the drainage tube can provide variable length configured with a continuously changing cross section allowing an accordion-like extension and/or collapse of the tube length, as shown in FIG. 2B.

In another aspect, drain tubes can have one or more spacers extending radially along their length, e.g., so that distal sections of the tube can not rest at a level below the collection bag inlet. For example, with a low, flat collection bag resting on the floor, a top center inlet port can be 3 inches or less off the floor. By providing one or more spacers with a 3 inch radius on the drainage tube near the bag, the tube can not contact the floor, or even come closer than 3 inches from the floor, so the tube can not make a dependent curl loop below the bag inlet level. A tube spacer can be, e.g., a length of foam tubing with thick walls and a lumen to receive the drainage tube. Optionally, the spacers can be disks with a center hole to receive the tube, or spheres with axial holes to receive the tubes.

In another aspect, the drainage tubes can incorporate an anti-reflux valve, to prevent reverse flow from the tube or collection bag to the catheter. Such valves can be one way valves, e.g., such as a reed valve or a ball and seat valve. The anti-reflux valve can be located at any position along the drainage tube, or optionally in association with the connection fitting with the bag or catheter.

### Drainage Collection Receptacles

Preferred drainage fluid collection receptacles of the invention have an aspect ratio greater in the horizontal than in the vertical. The receptacles are typically flexible bags, or more solid chambers, configured to have a low aspect ratio in use. Typically, the bags have more ceiling (top wall) surface and/or floor (bottom wall) surface than side wall surface. This configuration provides many benefits over old art vertically hanging collection bags, such as, e.g., the ability to rest securely on the floor, minimizing collected fluid depth (and thus minimizing pressures within the bag as it fills), lowering the height of the drainage tube inlet, and allowing the bag in use to be placed at any number of unobtrusive locations.

The low aspect ratio collection bags of the invention can be constructed in any suitable way. In preferred embodiments, the bags are fabricated from flexible plastic sheet or film materials. The body of the bag can be blown or spun in or around a coated mold as a single piece at once (e.g., as in the manufacture of latex gloves). In a preferred embodiment, the body of the bag is fabricated by fusing two sheets of material, one on top of the other, using heat or sonic energy, to provide a hermetically sealed common edge. For example, a round sheet of plastic can overlay a larger square sheet of plastic, and an appropriately shaped sonic welder can fuse the sheets together along the peripheral edge of the top round sheet. Optionally, a low aspect ratio cylindrical side wall can be welded or molded between the top sheet and bottom sheet.

The collection bag includes an inlet port to receive fluids from the drainage tube. In preferred embodiments, the inlet tube is located at the top of the collection bag. For example, the inlet can be at the center of the top wall of the collapsed (unfilled) bag. As the bag is filled the inlet will not experience the fluid pressures found at the bottom of the collected fluid. As the bag is filled, the top wall will typically take on a semi-hemispherical shape with the inlet at the highest point, or at least have the inlet float at the top of the fluid. The inlet does not have to be at the top center of the top wall, but it is preferred the inlet not contact positions where the top wall joins the bottom wall or side wall. It is preferred the inlet not be located where it is below the level of collected fluid in use with the bag resting naturally on the floor. In embodiments without fusion lines defining the joint between sections, it is preferred the inlet not be on a surface substantially vertical when the bag is substantially full of fluid, or on a surface that is below 60% or more of the fluid when the bag is substantially full. In preferred embodiments, the inlet port is located closer to the center of the top wall than to the peripheral edge or to the side wall. In preferred embodiments, when the collection bag is resting naturally on a horizontal surface (e.g., with the broader dimensions horizontal and the narrower dimension vertical) the inlet port is located above the majority of bag surface or above the bulk of any fluid present in the bag.

In some embodiments, the collection receptacle is solid enough to maintain a gentle vacuum against the external environment. These solid chamber receptacles are typically used to continuously evacuate fluids from inside an animal as they are produced. For example, solid chamber embodiments can be used to collect exudates from a chest cavity. As with the flexible collection bag, an inlet port receives fluids from a drainage tube. In preferred embodiments, the inlet tube is located at the top of the chamber. For example, the inlet can be at the top of the chamber. Because the chamber has a low aspect ratio, it is not easily tipped and has the inlet port relatively low level, in use. It is preferred the inlet not be located where it is below the level of collected fluid in use with the chamber resting naturally upright on the floor. In preferred embodiments, when the solid chamber receptacle rests naturally on a horizontal surface (e.g., with the broader dimensions horizontal and the narrower dimension vertical) the inlet port is located above the liquid collection volume, e.g., above the bulk of any fluid present in the bag.

The collection bag can include a vent. Some old art vertically hanging bags require ventilation to avoid the back pressure that necessarily builds up as they fill and press against the side walls. In preferred embodiments, the bag does not require a vent, but can accommodate inflow of drainage fluid without significant back pressure build up by expansion of the flexible bag structure (typically by raising the top wall).

The collection bags can include hatch marks (e.g., volumetric graticules) for ready measurement of accumulated fluids. For more precise measurements, the hatch marks can run from the peripheral edge toward the top or bottom wall center, to be read by picking up the bag from the opposite edge and allowing it to hang vertically. In this way the horizontal cross-section of the fluid is less and the vertical dimension greater for more volumetric resolution between marks.

In optional embodiments, the collection bags can include reinforced eyelets or other fixtures that facilitate hanging the bag. The mounting fixtures can be used to hold the bag while measuring collected fluid volume, as discussed above. The mounting fixtures can allow secure placement of the bag off the floor, e.g., while a patient is being moved in a wheel chair or when the patient's bed is being moved. Mounting fixtures can be located, e.g., on the periphery, top wall or side wall. It is often preferred the mounting fixture not be located opposite the fluid inlet (e.g., in the bottom wall). In many embodiments, the collection bag does not include mounting fittings for hanging the bag.

Many means exist to stabilize the position of the collection bags on the floor. Particularly when the bag has no collected fluid, it might slide out of position on the floor. To retain the bags in place one can provide, e.g., a weight stuck to or in a pocket on the bottom wall of the bag, sticky adhesive film on the bottom wall exterior, suction cups on the bag bottom, and/or the like.

Barriers can be provided to improve the sanitation and appearance of collection bags on the floor. For example, the bottom wall can extend peripherally beyond the fusion with the top wall to provide the look of a protective mat. Alternately, the bags can be placed on a mat, in a rack, or in a tub, e.g, to physically isolate the bags from contact with the floor.

### Urometers

The drainage collection systems of the invention can include a urometer to monitor the rate and/or quantity of drainage fluid collected. Typically, the urometer can include a chamber with at least a section of wall visible on the exterior of the drainage collection system. The visible wall can be transparent or translucent so the internal level of fluid can be viewed against volume indicating graticules on the wall.

In certain embodiments, the urometer is a transparent chamber with graticules on outside, and surrounding or adjacent to main waste holding chamber. The urometer chamber has at most 10% the volume of the main chamber. The drainage tube flows into the urometer chamber so that the rate of fluid drainage with time can be determined by noting the progress of fluid levels against the volumetric graticules. To empty the urometer, or to re-zero the inflow fluid level, the fluid can be transferred to the main chamber, e.g., by pouring through a port of conduit.

In one aspect, the urometer component of the system can completely surround the main collection chamber. The urometer compartment can be in fluid contact with the main collection compartment through a port or conduit. The port or conduit can include a manually controllable or one-way valve allowing fluid to controllably flow from the urometer to the main collection compartment.

Alternately, the urometer can be positioned beside the main collection chamber with a common wall between the urometer and the collection chamber. After measurement in the urometer, fluids can be poured into the main chamber, e.g., through a port at the top of the common wall.

The main chamber can optionally include a fluid collection bag, e.g., as described above. For example, the main chamber can include a flexible collection bag having horizontally opposed top and bottom walls. The space between the top and bottom walls can expand as incoming fluid enters from the urometer through a top center inlet.

In an optional embodiment, a source of intermittent or constant suction (from a motorized bellows-suction pump or other such generic suction pump) could be interfaced with the top-most portion of the urometer, such that intermittent (or constant) suction (gentle to strong) could be delivered to the bladder or other body space being drained. The source of suction could be automated and run on a timer.

### Methods of Collecting Fluids

The present methods of collecting biological fluids generally comprise placing a catheter at the source of a fluid drainage from an animal, providing a low aspect ratio collection bag at a level below the catheter, providing a drainage tube between the catheter and collection bag, and allowing the fluid to passively drain from the animal into the collection bag under the influence of gravitational force.

Animals can be catheterized, as is known in the art. Urinary catheters, such as Foley catheters can be inserted into the urethra to enter the bladder. A small “balloon” at the proximal end of the catheter can be filled with a fluid, through an auxiliary conduit to the exterior, in order to prevent the catheter from slipping out from the bladder. Wound catheters can be as simple as a flexible plastic, rubber, or silicone rubber tube inserted through the wound opening.

Draining the fluid from the animal to the collection bag can be as simple as providing a drain tube that ultimately runs from the catheter to a collection bag at a lower level. It is preferred that the drainage tube be just long enough to traverse the distance down from the catheter to the collection bag. It is preferred that the drainage tube be positioned so that no section along the tube is between two higher tube sections. It is preferred that the tube be positioned so that no section along the drainage tube is below the level of the input port where the tube flows into the collection bag. To minimize the possibility of developing a dependent curl loop in the drainage line, it can be configured with a means of varying the length so that not enough tubing is available to contribute to a dependent curl. To prevent a drainage tube near the floor and bag from dipping below the bag inlet port, spacers can be provided below or radially extending from distal sections of the tube near the bag.

Collection bags can be provided as described in the Drainage Collection Bags section, above. The collection bags can be provided with a flexible top wall and/or bottom wall, so that the bag expands predominantly in the vertical as it fills, rather than in the horizontal. The collection bags can be provided with a broad flat aspect ratio and with an inlet port positioned above a smaller (e.g., smallest) dimension of the bag. For example, the inlet port can be on a top surface of a bag having a height less than width and/or horizontal depth, when the bag is empty and/or when it is full.

The collection bags can be provided resting on a horizontal planar surface, such as a floor or mat on the floor. The bag can be placed directly below the catheter, or directly below where the drainage tube hangs over the edge of a bed where the catheterized animal is resting. Optionally, the bag is placed laterally offset so that the drainage tube drops to the bag at an angle from vertical, but preferably never passing through the horizontal. To positionally stabilize the bag, it can be weighted or stuck with an adhesive to a desired location on the floor.

It is preferred the collection bags of the invention not be hung. For example, it is preferred the bags in use not be mounted to dangle from a high point, e.g., without lower support from resting on a horizontal surface.

### Examples

The following examples are offered to illustrate, but not to limit the claimed invention.

### Example 1

### Collection Bags

Collection bags have been designed that provide features configured to minimizing backpressures in the drainage tube (and ultimately the catheter). The collection bag is a plastic bag of round or rounded-triangle shape. This bag is designed for use lying flay on the ground. The drainage tube can be similar in design to currently available products and can possess non-proprietary features, such as, e.g. urine sampling port.

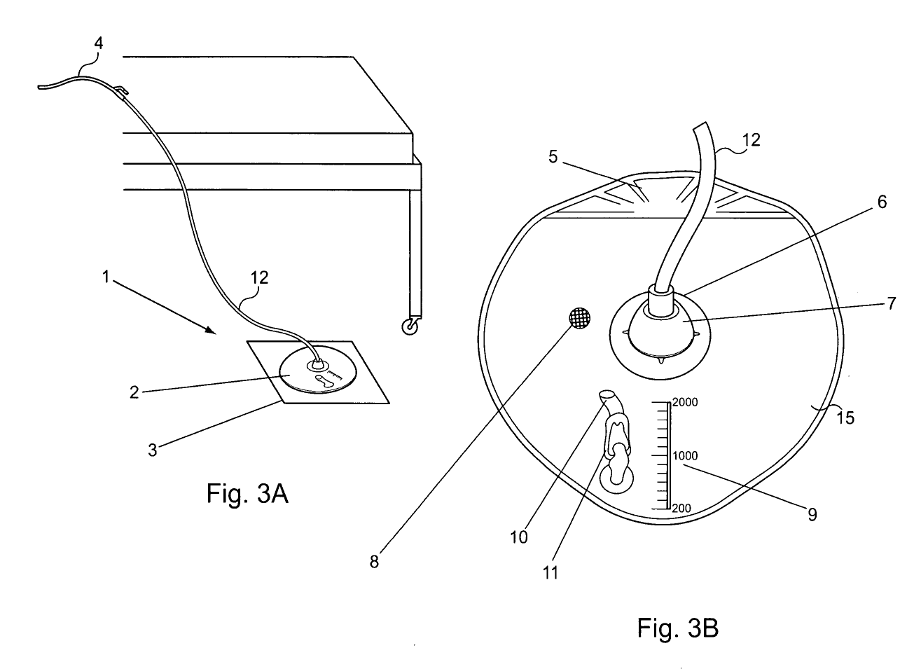

The following is a description of a proposed “flat” drainage bag, e.g., as shown in FIG. 3A. The proposed fluid collection system 1, used as intended, lies flat on ground. That is, e.g., the proposed device is designed with a drainage bag to be used laying in a flat position, on a horizontal surface, such as the ground, a floor, on a dedicated “pad”, basin resting on the floor, and/or the like. The drainage collection bag 2, can lay flat directly on the horizontal surface or on a clean “drainage bag pad” 3. In use, catheter 4 is typically located above drainage tube 12, which feeds into collection bag 2.

FIG. 3B shows a close-up view of some of the proposed features of the proposed flat drainage collection bag. When the operator wishes to measure the urine volume that has drained into the bag, he/she can hold the bag vertically by the reinforced bag holding tab 5, so that the urine layers on the opposite side, where volume “hatch marks” 9 are located on top wall 15. The volume of fluid in the bag can be read directly from the position of the urine meniscus along the hatch marks.

The drainage tube connects to a hard plastic fitting 7. At the site of this connection, the drainage tube 12 can be made of, or simply re-enforced by, a sleeve 6 of flexible plastic (e.g. silicone), to allow the terminus of the drainage tube to bend without occluding by kink, to better accommodate an oblique trajectory often traveled by the drainage tube into the bag positioned flat on the floor. Here, the drainage tube terminates 2-4 cm above the bag at the dome of this inlet fitting.

An air vent 8 hole in the upper surface of the bag is covered with a hydrophobic fine mesh, to allow air to escape from the bag, while preventing leakage of urine from the vent itself. This is a standard feature of drainage bags which may or may not be present in embodiments of the present inventions.

Drainage bag tube-outlet 10 allows fluid contents to be emptied from the drainage bag, when desired. Standard “snap” clamp 11, can seal the outlet when it is not in use for drainage of the bag.

The connection between the drainage tube and the bag is unique compared to the current state of the art in that it is designed to maximize a vertical (not perpendicular) connection between the bag and the drainage tube, while minimizing the risk that torque applied to the bag by the drainage tube will “flip the bag over”. While the drainage tube is usually clear flexible plastic, the end of the tubing closest to the bag connection point can be made of silicone rubber to allow for greater “joint-like” flexibility at the connection point. Alternatively, the drainage tubing can be made of all silicone, if desired.

The junction of the tube with the bag can be located anywhere on the bag, but, if no anti-reflux valve is incorporated into the design of the final product, then the junction should be located in the center of one face of the bag's triangular (or “triangular-like”) shape, so that when the bag is picked-up and positioned vertically (for example, to allow measurement f the bag's fluid content), the bag's fluid content does not reflux into the patient. Hence, positioning the tube entry point to reside diametrically opposite to where the fluid will be forced to collect during measurement minimizes the risk of unintentional forced reflux into the patient.

The connection point from the tubing toward the bag can either be directly into the bag, or, the tubing can connect to an inlet filling, which opens into the bag. Within the inlet fitting, the interface between the bag and the tube can, if desired, be fitted with an “anti-reflux” valve, such as a flat “heimlich-type” valve, or, a flap valve as is currently standard. The clinical need for any valve is debatable, but one can incorporate an anti-reflux valve if desired.

The bag drainage outlet can be based on any number of regulated outflow spigots now or past in use. Examples include a stopcock, an outflow “nipple tube” that is bent closed with a clamp when the bag is to remain full, and unclamped when bag drainage is desired. Other examples include all manner and method of simple outflow valves available.

The top side of the bag can be fitted with hatch marked numbers to measure volume within the bag. The hatch marks can be oriented on a given side of the bag, so that to measure bag volume, the operator holds the bag up vertically, so that the fluid contents collect dependently within the bag, so that the fluid surface lies perpendicular to the volume hatch marks. If the bag shape is “triangular”, then the operator can be instructed, when measuring contents, to hold the bag vertically with the point corresponding to the measuring marks toward the ground.

It is recognized that using volume-hatch marks on a container to measure that container's fluid volume can be most accurate when the meniscus of the fluid is as small as possible. With the collection bag oriented flat on the ground, the meniscus is actually a very large area. To measure the bag's contents, the operator can be instructed to reposition the bag into a vertical position, with the drainage tube held vertically (pointing away from the ground) from the inlet point with the hatch marks directly opposite, e.g., preferably printed onto a cone-shaped triangle-corner of the bag.

The bag can be weighted. When the bag is full of urine, such a weight is typically not necessary, but when the bag is completely empty, and weighs very little, it may not stay easily where the operator positions it on the ground. Moreover, “memory” in the plastic tubing may cause the bag to curl slightly, and tip over, or, to “pull” easily closer to the patient. In some embodiments, the underside (bottom) wall of the bag can be fabricated with a “jacket-flap” pocket (open to the outside of the bag), so that, if desired, one can fit a proprietary “weight” into this pocket, to force the bag to remain in place a set distance from the patient's bed in order to maintain a maximally straight-downward oriented drainage tube at all times. The “weight” can be provided separately, and consist of as little as a plastic covered thin lead or metal disk, of minimal overall weight sufficient to hold the empty bag fixed on the ground. The bag's plastic coating can allow it to be easily washed and reused. Alternatively, the “weight” can consist of a separate flexible chamber that is filled with tap-water by the operator, and then inserted into the bag underside sleeve-pocket to weigh down the collection bag in place.

Alternatively, the bag can be secured flat onto the ground using “sticky pads” on the underside of the bag. At the appropriate time, these can be peeled to expose their sticky surface, and used to affix the bag to the ground.

An alternative design solution, e.g., to help maintain the empty drainage bag on the ground wherever desired, consists of suction cups attached to the underside of the bag. One or more “suction cups”, when wet and affixed onto the floor, will serve to anchor the bag wherever desired. The suction cups can be manufactured already on the bag, or, provided separately and fixed onto the bag, e.g., via a pre-made snap or VELCRO™ connector.

A key feature to note is the horizontal shape of the proposed overall bag design is that a “flat-use” design is not subject to the increased pressure resulting when, for example, a vertically oriented “flat bag” is filled. For example, imagine two identical closed system (non-vented) pancake shaped bags, each filled with 10 cc of air, then steadily filled with a set volume of water. One bag is placed flat on the ground as it is filled, and the other is hung vertically (i.e., flat shape perpendicular to ground). The pressure of the 10 cc air will be higher in the vertically oriented bag as it fills, as compared to the identical flat-positioned bag. This is because, e.g., as the vertically oriented bag fills, its shape assumes a triangle, with fluid collected on the bottom of the triangle. The sides of the triangle are stressed by the weight of the bag, and the stress increases pressure of all fluids inside.

It appears that all leading drainage bag products in the US (which not coincidentally, are vertically oriented in use), have an air vent at the top of the bag, to allow for air-pressure release. Hence, as the flat-use bag is an inherently lower pressure system, it lends itself to the possible use of completely closed drainage bags, without air-vents. A ventless bag does have one preferred requisite: that the bag itself must be a low pressure, airless system. Hence, when the bag is opened for initial use, the bag should be substantially empty of air before it is connected to the patient (accomplished with clear instructions, and pre-packaging in a flat folded square, or circle).

Furthermore, if the bag is completely closed, and has no air vent, and has no hooks, etc, it is better adapted as a disposable product. For example, in Europe, disposable urinary drainage bags are used, but the design of these bags reflects that they are designed to be used in a vertical, “hanging” position. These are made of a light-weight plastic (highly compliant, but with good tensile strength). To our knowledge, none comes fitted with a urometer, and none is specifically designed to be used on the ground. The present designs are different from these in that they are designed to be used flat (e.g. the connection of the drainage tube to the bag reflects this, etc). Further, the present bags can optionally be fitted with an air vent (e.g., on the hard plastic housing and/or urometer), to resemble the types of bags sold in the USA.

The present bag designs can be fitted to function with any number of anti-reflux valve designs. The simplest is a flap-valve similar to the “Heimlich valve” design used in many “urinary drainage leg-bags”: at the tubing bag junction, all fluid is forced to pass through two leaves of plastic which are connected at their sides, such that together they comprise a circumferential tube through which the inflow fluid passes. The plastic leaf members are much longer than they are wide, such that they form a compressible tube, whose end resides in the lumen of the bag. Furthermore, the leaves are in direct apposition when no fluid is flowing between them, and they widen just enough to allow fluid to pass between them. If the bag is accidentally stepped on, to avoid dangerous sudden high-pressure reflux into the patient's bladder via the drainage tube/catheter, the flap/Heimlich valve serves to impede retrograde flow: as pressure suddenly increases in the bag, pressure on the outer walls of the flap valve forces the leaves to come together, preventing reflux through the flap valve. This “Heimlich-like” flap valve can be interposed between the inflow tube and the bag at the junction of the two (when a urometer is not present), or, it can be interposed between the urometer outflow hole and the lumen of the bag when a urometer is present.

Alternative flap-valve designs are feasible. For example, the inner surface of the bottom-side of the bag just below the inflow tube bag junction (inflow housing) can serve as a flap valve to close access to the inflow tube lumen. To achieve this, either the angle that the inflow tubing makes relative to the bag as it joins the bag must be less than 90-degrees, or, the angle remains close to 90-degrees, but instead, a separate flap of plastic (area approximately double area of inflow tube lumen) can hang from the inner surface of the dome of the bag (or from the inner surface of the inflow tube housing). This “flap” can hang low enough to allow inflow to proceed unobstructed past it under normal use. However, with a sudden pressure increase in the bag (e.g. someone steps on the bag accidentally), the flap is forced “upward” toward the inflow tube, and occludes the inflow tube. This flap can be made of any appropriate synthetic whose properties facilitate such function.

Lastly, another proposed antireflux design is a ball-valve design, whereby a ball-valve lies within the tubing housing, and when pressure within the bag increases, the hollow plastic ball is forced “upwards” toward the inflow tube, occluding its lumen to protect the patient.

### Example 2

### Urometers

Urometers can be integrated into drainage collection systems of the invention. In preferred embodiments the urometer is integral to the collection bag. For example, the urometer can be a smaller chamber associated with the main collection bag to first receive drainage from the tube and having volume graticule hatch marks for reading fluid volumes received.

Urometers can be fabricated from a hard clear plastic, or from a soft flexible plastic that distends upwards as the urometer is filled. When the user wants to visually measure the fluid content inside (soft urometer), the bag is manually suspended by the top of the urometer so that the fluid fills uniformly the urometer chamber. Hatchmarks indicate the volume within the urometer.

An exemplary urometer design can be “a cup within a cup”, where the drainage tube connects to the top of the urometer cup, and fluid collects inside the urometer. When the urometer cup is sufficiently filled, the cup is tilted over, so that the fluid in the urometer drains through the drainage hole that connects the inner cup (urometer) to the outer cup (the bag). The urometer contents are thus emptied into the bag, e.g., through a common port or conduit. The walls of the urometer cup is made of hard plastic or thin flexible plastic. The outer walls of the cup are open to the interior of the drainage bag.

Fluid volume within the urometer cup can be measured by volume hatch marks located along the wall of the cup. The roof of the cup is sealed closed by an arched dome of hard clear plastic. The dome connects to (and opens into) the drainage bag. Close to the roof of the urometer cup, there is a large round or oval shaped opening (“urometer drainage hole”) on one side of the cup, which allows the contents of the cup to drain out of the cup and into the drainage bag.

If desired, the urometer can be sub-compartmentalized, such that collected fluid first fills a smaller subchamber (not illustrated). This subchamber serves to allow more frequent measurement of inflow (e.g. total volume is approximately the normal urine output in 30 min). The first subchamber is designed to fill (by overflow) into a second larger subchamber, which serves to yield patient fluid output measurement over longer intervals (e.g. total volume is approximately equal to normal urine output over 60 min). Finally, the second subchamber can drain into either another larger subchamber, or drain into the main collection bag itself (so that fluid output can be measured over even longer time intervals, such as every 12 hrs, etc).

When the operator wishes to measure a urine output over a time interval, the urometer is examined and volume recorded, just as with all current urometers. Next, the operator “zeroes” the urometer by emptying its contents into the bag, simply by tilting the “cup” part of the urometer to pour the contents out of the urometer drainage hole, into the bag.

Note that when the urometer is made of hard plastic, the urometer is effectively designed to operate as a “vertical” structure (in that incoming fluid is causes the fluid meniscus within the urometer to rise vertically, in accordance with gravity. If the urometer is made of thin flexible plastic, the urometer fills mainly horizontally, as the flexible walls of the urometer are displaced laterally before the become meniscus rises. The drainage bag is designed to operate in a “flat” horizontal configuration. Ultimately, the proposed urometer drainage bag device can be a combination of a vertical urometer and a horizontal bag.

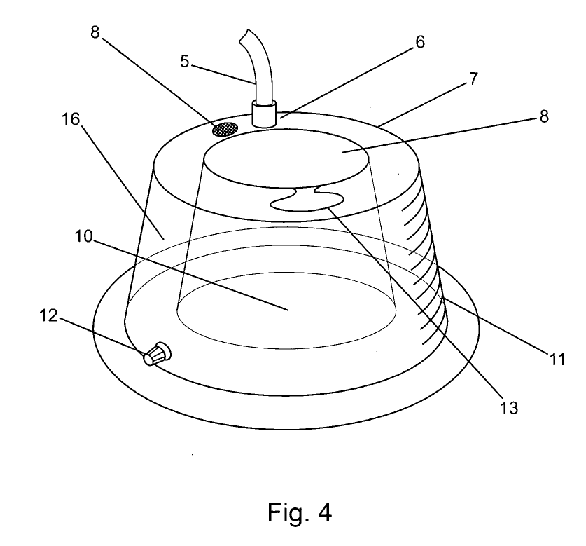

FIG. 4 shows a drainage collection bag including an outer urometer chamber. In this design, drainage tube 5 terminates at the upper surface of the urometer 6 to drain in through the hard plastic upper surface of the urometer 7. At the site of this connection, the drainage tube can be made of, or simply re-enforced by, a sleeve of flexible plastic (e.g. silicone), to better accommodate an oblique trajectory of the drainage tube into the bag positioned flat on the ground and to prevent occluding kinks in this part of the tube.

The drainage tube opens into the double-walled lumen 16 of the urometer. The space defined by the inner walls of the urometer is covered by a plastic dome 8. The space 10 beneath this dome, within the inner space bounded by the inner walls of the urometer lumen, opens to (and is contiguous with) the interior of the drainage bag.

As the urometer fills with urine, the urine volume can be measured using the volume hatch marks 11 printed vertically on the surface of the urometer housing. The urometer sampling port 12 can be fashioned in any way, but a simple embodiment is a screw-open luer-tip valve, which can be attached to a syringe. When desired, a sample of the urine freshly-collected within the urometer can be collected, for analysis.

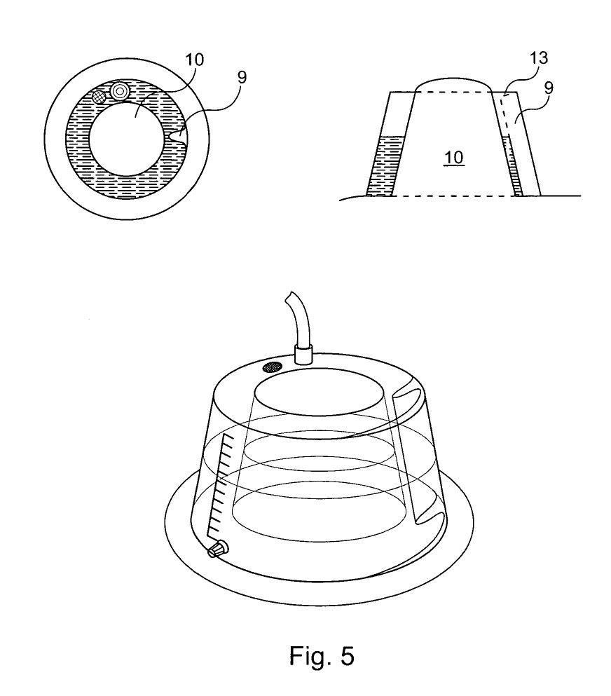

When the Urometer is full, or, when the operator wishes to initiate a new collection period (or urine sample), the urine that has collected within the urometer can be drained into the drainage bag by tilting the urometer so that the urine drains through the urine outlet 13, and into the lumen of the drainage bag 10. A potential challenge will be to get the urine to drain into outlet 13 when the urometer is tilted. An alternative flow path for the urometer outlet is a conduit 9 on the inner surface of the urometer outer wall, as shown in FIG. 5. In this embodiment, the urometer can be tilted towards the concave surface of the outer wall, where the alternate location of the “urometer to drainage bag” port would lie. As urine collects in this concave space, it can drain into the lumen of the drainage bag by entering the urometer to drainage bag port 13 located close to the roof of the urometer lumen. The urine will enter the port, and drain into a segregated “shaft” (conduit 9) that connects the urometer to drainage bag port 13 to the drainage bag lumen 10.

### Example 3

### Other Design Aspects

The fluid drainage systems can include additional features to enhance performance.

I. Plastic “elevator balls” (disposable, foam or biodegradable Styrofoam, or other material) that can be snapped onto the drainage tube anywhere along its length, to maintain the drainage tubing at a minimum height from the ground. Each ball can be solid or hollow or partially hollow, etc. Each ball can possess a partial-thickness slit or groove to allow the drainage tube to fit snugly into the ball. Each ball need not be strictly spherical in shape. The purpose of the ball is to provide clearance beneath the drainage tube so that the drainage tube retains a minimum elevation. The elevator ball would most typically be used close to where the drainage tube joins to the bag, where it would otherwise be more likely to “dip” with gravity, below the level of the juncture between the tube and bag.

II. “Telescoping tubing” is a means by which redundancy in tubing length can be eliminated by simply telescoping the tubing into itself. Telescoping tubing could either be added to some or all drainage tubing products, or could be marketed as an accessory product, which the healthcare professional can interpose into position between the drainage bag and the patient's catheter/tube/body. The design of “telescoping tubing” would be such that the diameter of the tubing would be graded to steadily increase, either toward the bag from the patient, or toward the patient from the bag. In either case, the redundant segment of tubing is made to telescope into the closest segment of tubing of greater diameter. Whether the final telescoping is toward the patient side or toward the bag side is depends on operator preference and what the viscosity of the fluid being drained. Some fluids that are thick, or tend to clot, would likelier drain better when the telescoping occurs with wider diameters toward the collection bag.

III. Another novel design feature is the concerted attempt to make a disposable version of my flat urinary drainage bag. It should be noted that the leading manufacturers' urinary drainage bag products marketed in the USA are designed for relatively long term re-use within the same patient: they have re-sealable drainage valves, for example. Several specific features can be incorporated, separately or altogether, to yield a more disposable product:
a. The bag may be made with thinner and cheaper plastic material. A thinner material is feasible because the bag itself doesn't hang. Hence, the tensile strength necessary in the bag wall material is less for the present flat collection bags.
b. Because the present collection bags don't hang, they does not require the expense of manufacturing hanging hooks, ties, or special seal moldings at the top of the bag element for attachment of the hooks.
c. The present inventive collection bags can be made without a drainage outflow valve. The bag may be designed as described in the paragraphs above, but without a drainage system. When the bag fills, passively, it is discarded. As such, there are no moving parts (hooks, ties, or valves), rendering a more disposable device.

IV. There is wide prejudice, on the part of hospital nurses, against placing a urine drainage bag directly on the ground. Such nurses believe that if the bag must be placed flat on the ground, then it should at least be placed into or onto a “clean” surface other than the ground. In light of this, the floor resting collection bags can be used directly on a flat surface other than the ground. For example:
a. A flat urinary drainage bag stand can be provided, on which to lay the flat urine drainage bag. The stand can resemble a flat step, whose flat surface height is 1 cm to 1 foot off of the ground, but not so high that a slack in the drainage tubing is creating, leading to the formation of a dependant curl.
b. A flat basin can be provided into which the flat drainage bag is deposited. The floor of the basin serves as a barrier between the bag and the floor. The walls of the basin may be high (like a bucket or plastic bowl) or low, essentially providing a holding “plate”.
c. A trash-can like receptacle can be provided into which the drainage bag is deposited for the purpose of eliminating dependant curls along the drainage bag tubing: the receptacle's high walls maintain the drainage tube above the bag at all times. The receptacles high walls are the salient feature, not the floor of the receptacle.
d. A flat “pan” (plastic circle or square) which can be sprayed with alcohol disinfectant by the nurse for re-use between patients or be disposable.
e. A disposable paper, plastic, or other synthetic soft flexible material that is placed beneath the proposed flat drainage bag (e.g. a disposable plastic liner, or a “doily”, etc.).
f. A “peel-away” liner can be affixed to the proposed flat drainage bag. The outer surface of the peel-away surface may be coated with bacteriostatic chemicals.
g. All devices above may also be completely or partially coated with a bacteriostatic agent.

### Example 4

### Drainage Collection System With a Solid Chamber

The drainage collection systems of the invention can include collection receptacles capable of holding a gentle vacuum. Such systems can include many aspects of the flexible bag systems, discussed herein, but have a relatively solid (more firm, less pliable) collection receptacle.

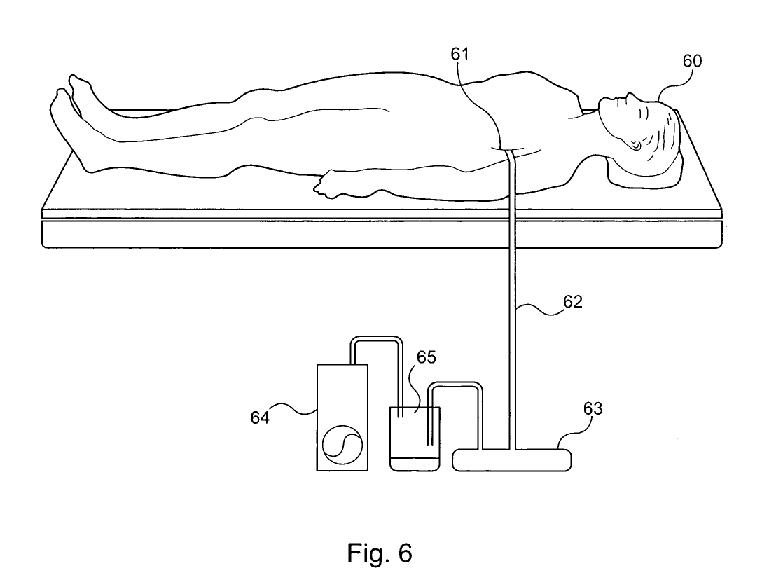

FIG. 6 shows a patient 60 having an incision through which a chest tube 61 has been inserted to collect fluid from the chest cavity, e.g., the plural space. A drainage tube 62 runs directly to a low aspect ratio solid collection chamber 63.

In optional embodiments, the collection chamber is partially evacuated of air using a vacuum pump 64, e.g., through a fluid trap 65.

### Example 5

### Management of Hydrothorax, Hemothorax and Productive Pneumothorax

Treatment of pneumothorax requires drainage of gasses and fluids from the chest cavity, and application of a negative pressure in the chest cavity to keep the lungs inflated. For example, treatment of pneumothorax can include insertion of a catheter into the chest wall for application of a vacuum −20 cm water.

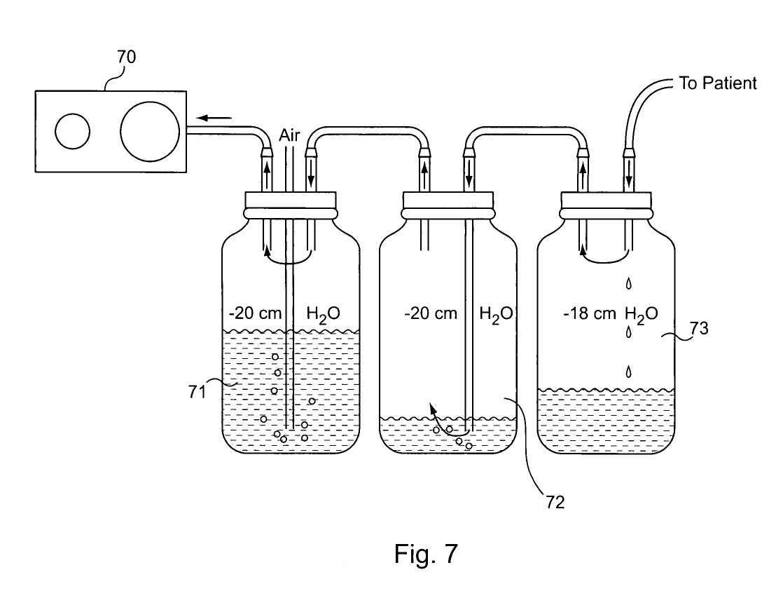

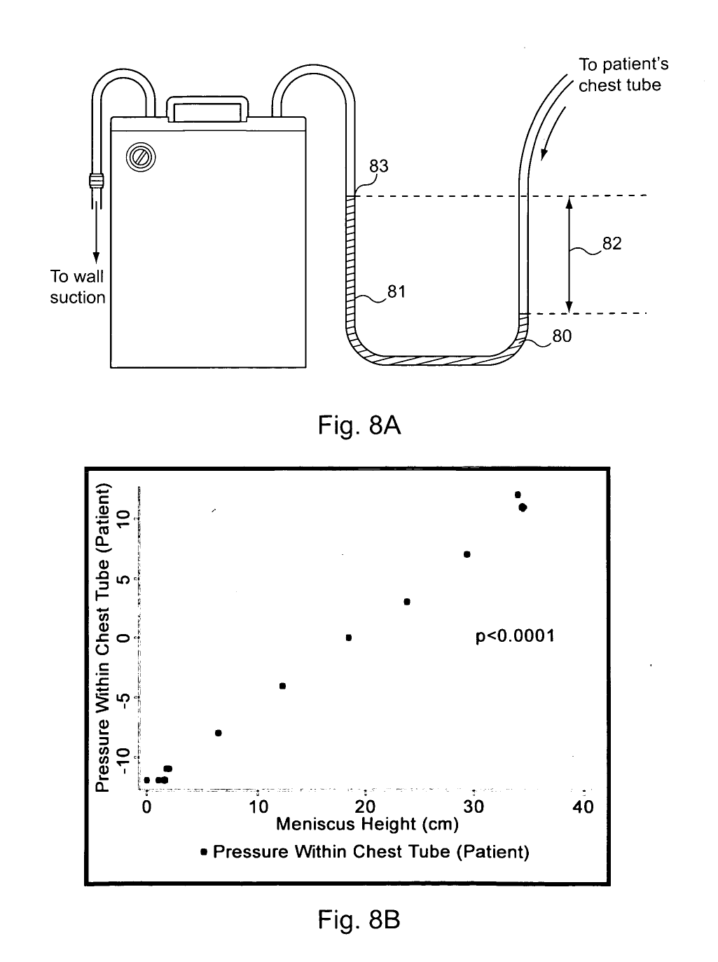

Vacuum can be applied from a vacuum pump through a three-bottle collection system to the patient, as shown in FIG. 7. Modern embodiments typically incorporate the vacuum pump 70, pressure control 71, sealing 72 and collection 73 components into a single unit, as shown in FIG. 8. However, because of the height in the units, the patient can experience less vacuum, or even positive pressures, should fluids collect in the loop offered by the tall collection systems.

We have shown, as depicted in FIG. 8, that as fluids collect in a dependent loop offered by systems, such as the PLEUR-EVAC™, the designated −20 mm Hg vacuum is only provided through a fluid free loop. As fluid 80 collects in dependent loop 81, the draw of the vacuum pump is countered by a height differential 82 between the fluid menisci 83. The chart of FIG. 8 shows that a dependent loop the height of the system can actually result in a positive pressure in the patient's chest tube. Such a pressure can negate or reverse the intended benefits of the vacuum therapy.

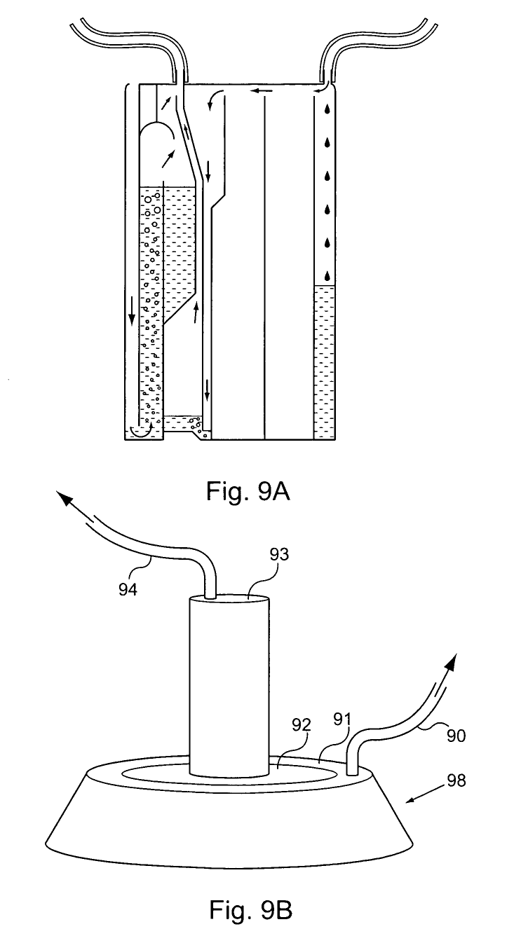

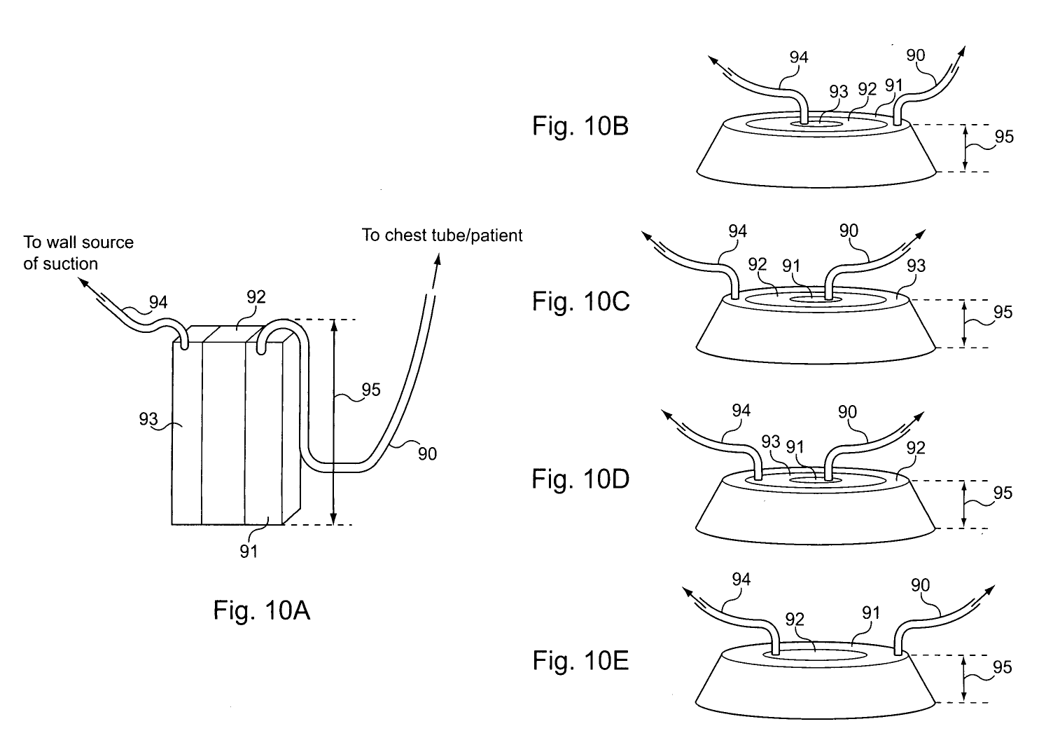

To avoid the development of dependent loops in suction systems, we have designed a system wherein at least the sealing component and collection component are provided in a low aspect ratio device. For example, FIG. 9 shows standard collector unit 97 chambers and channels that can be incorporated into a low aspect ratio embodiment 98. FIG. 10A shows a schematic of the prior art high aspect ratio three-bottle system. FIGS. 10B to 10E are schematic diagrams of alternate systems. In each case, the suction control component 93 can have a somewhat taller height than the other compartments, or all the other compartments can be taller, e.g., at least 20 cm, e.g., to allow −20 cm H₂O vacuum control. The exemplary systems concentric low aspect ratio chambers with the benefits of increased stability resting on the floor, and reduced maximum dependent loop height. Chest tubes 90 to the patient are in fluid contact with a fluid collection chamber 91 through an inlet port. Water-seal chamber 92 is in fluid contact with the collection chamber through a dip tube with an outlet tube configured to prevent collected fluid overflow from traveling further toward the vacuum pump, as known in the art. Optionally, the systems can include a suction control component 93. Finally, the system can include a suction tube 94 to a vacuum source. Optionally, the vacuum source can be incorporated into the fluid collection unit.

The height 95 of the collection unit, measured from the bottom (that rests on a surface, in use) to the chest tube inlet is preferably less than 35 cm, less than 20 cm, less than 15 cm, or less than 7 cm. In a most preferred embodiment, the height is 5 cm or less. The aspect ratio (height to system base width) is preferably a low aspect ratio, e.g., 0.5 or less, or 0.25 or less.

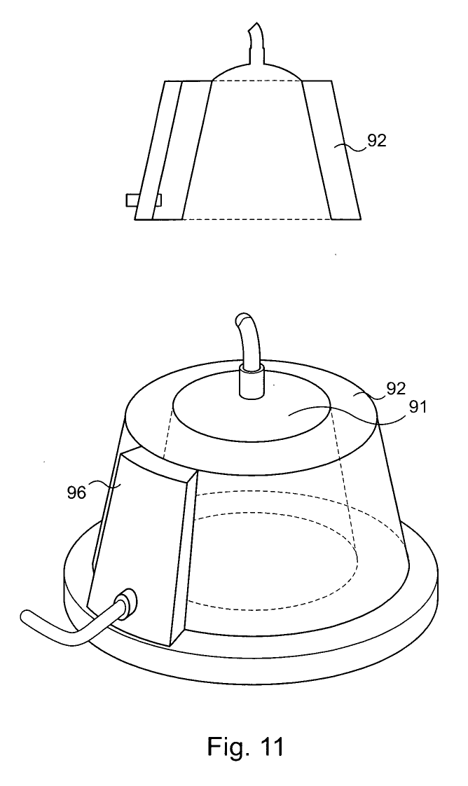

FIG. 11 shows an exemplary drainage device wherein the vacuum pump or suction control chamber 96 is mounted onto a structure comprising a short fluid collection chamber and water seal chamber.

Should a dependent loop be present, pressure within the proximal tubing segment increases (becomes more positive). If the dependent loop is of sufficient height, the pressure can increase, e.g., from −20 cm H₂O to 0 cm H₂O when the loop contains −25 cm³ fluid. Since standard chest drainage device tubing holds 1 cm³ per cm length, it follows that when a dependent loop of at least 25 cm height is filled with fluid, pressure within the tubing segment residing within the patient increases to ˜0. If the loop height is >25 cm, and the tubing is allowed to fill beyond 25 cm³, then, it is possible for pressure within the proximal tubing can increase above 0 cm H₂O. If this occurs, one proposed solution is placement of a one-way air valve (see Example 7, below) at the proximal-most segment of tubing (e.g., where it connects to the chest tube). This air valve can allow release of positive-pressure (>0 cm H₂O) air to the environment, and allow the suction chest drainage system to re-establish more negative pressure within the tubing proximal to the dependent loop.

### Example 6

### Tube Coiling Collar

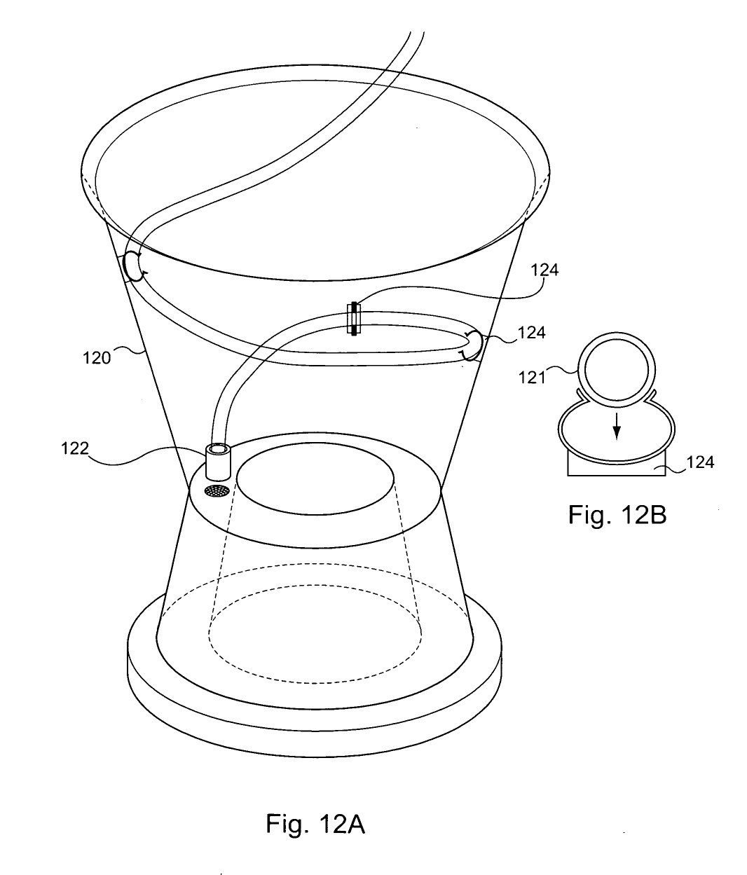

A conical collar can be provided to route a drainage tube in a downward spiral to a drainage device inlet. The collar can have a conical interior surface with increasing radius toward the upper opening. As shown in FIG. 12, a collar 120 can be mounted above the drain tube 121 inlet 122. The conical shape of the collar interior can provide support for the drain tube to naturally coil upwards. As the distance between the patient and the drainage device changes, more or less of the drain tube can coil into the collar, without formation of a dependent loop. For example, as additional tubing falls into the collar, there is a tendency of the tube to coil along the wall above or along side lower tubing because it is lower energy to stay against the wall than to climb in over a lower segment of tubing. Thus, the proximal tubing above tends to stay above and not fall into a dependent loop, even as more tubing is fed into the collar.

In some embodiments, the inside surface of the collar can have resilient mounts to receive excess tubing in a downward coil. For example, “snap” fasteners 124, as shown in FIG. 11B, can be mounted in a pattern defining an up spiraling coil along the inner surface of the collar to hold the drainage tube in a desired spiral orientation. Depending on the amount of excess drainage tube, more or less tubing can be captured in the fasteners along the inner collar wall.

### Example 7

### Drain Tube Pressure Release

In an embodiment of the inventions, a one-way pressure relief valve can be incorporated into a drainage tube to prevent accumulation of back pressure, e.g., from the presence of a dependent loop.

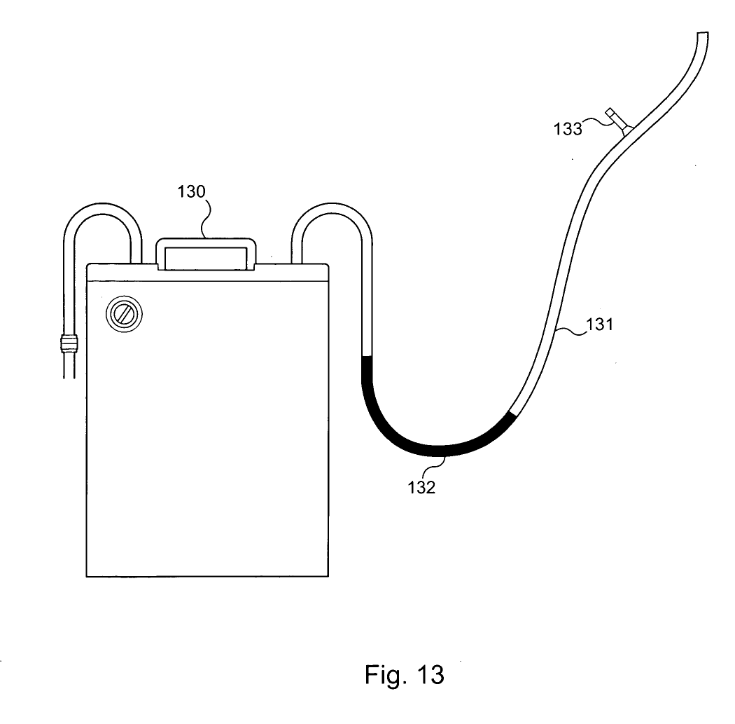

FIG. 13 shows a drainage tube collection unit 130 having a drainage tube 131 to patient. The drainage tube has developed a dependent loop 132 with a meniscus height differential. In order to prevent a back pressure in the proximal end of the tube, e.g., when the meniscus differential is allowed to relax, one-way pressure release valve 133 is provided in the drainage tube.

The one-way valve can be of any suitable type, e.g., diaphragm, ball valve, reed and/or the like. The valve can be fitted onto the proximal-most end of the drainage tubing, e.g., close to where it receives either the urinary catheter or the chest tube, from the patient. The one-way valve would thus allow positive-pressure air to escape from the lumen of the drainage tube. The one-way feature would not allow air to enter, thus preserving a negative pressure, e.g., should it be desired to suction a wound.

It is preferred that the one way valve be positioned to point upward (opposite gravity), to reduce the likelihood the valve would come into contact with a liquid in the tube. The one-way valve can be covered with a sealed bubble chamber or stuffed with wadding to prevent liquid from escaping past the valve. Optionally, the one-way valve can be vented to the collection unit via a conduit.

The one-way valve can be integrated into the drainage tube. Alternately, the one-way valve can be a separate unit, e.g., configured to be inserted in the tube between the catheter section and collection section.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, many of the techniques and apparatus described above can be used in various combinations.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A device for collection of biological fluid drainage, wherein the device comprises:
a bag comprising an inner space between a top wall and a bottom wall, wherein the inner space is characterized by a width or a depth greater than a height; and,
a drainage tube in fluid contact with the bag inner space through an inlet port located in the top wall;
whereby in use the biological fluid flows from the drainage tube into the bag inner space.

2. The device of claim 1, wherein the top wall and bottom wall are substantially planar and parallel when the inner space is empty of fluid.

3. The device of claim 1, wherein the top wall or the bottom wall consist of a flexible polymer sheet.

4. The device of claim 1, wherein the top wall and the bottom wall are in direct contact at a hermetically sealed peripheral edge of either the top wall or the bottom wall.

5. The device of claim 4, wherein the inlet port is not in direct contact with the peripheral edge between the top wall and bottom wall.

6. The device of claim 1, wherein the inlet port is entirely surrounded with a planar surface of the top wall.

7. The device of claim 1, wherein the top wall and bottom wall are in direct contact with each other.

8. The device of claim 1, wherein with the width of the inner space is greater than the height of the inner space when the bag is full of the biological fluid and resting with the bottom wall in contact with a horizontal surface.

9. The device of claim 1, wherein the inner space is not vented to the external environment in use.

10. The device of claim 1, wherein the bag is configured so that there is less tension on the bag walls with the device resting on a horizontal surface with the inlet port uppermost than with the device resting on a horizontal surface with the inlet port positioned laterally.

11. The device of claim 1, wherein the drain tube or inlet port comprises a one way valve configured to allow fluid flow into the bag but not out of the bag.

12. The device of claim 1, further comprising spacers mounted around the drainage tube, thereby ensuring the tube is spaced from contact with adjacent surfaces.

13. The device of claim 1, further comprising a barrier under the bottom wall, thereby preventing contact of the bottom wall with a surface the device rests upon.

14. The device of claim 1, further comprising a weight, suction cup or sticky surface mounted to an external surface of the bottom wall.

15. The device of claim 1, further comprising a collar mounted to the device above the inlet port, which collar has a conical inner surface expanding upwards.

16. The device of claim 1, wherein the drain tube is configured have a telescoping length.

17. The device of claim 1, wherein the bag is other than a bag comprising a mounting device for hanging the bag.

18. A urometer comprising:
a first chamber having a first volume and mounted within a second chamber in fluid contact with the second chamber through a conduit or port;
wherein the volume of the second chamber minus the volume of the first chamber is at least 10-fold less than the volume of the first chamber.

19. The urometer of claim 18, further comprising a drain tube in direct fluid contact with the second chamber.

20. The urometer of claim 18, wherein an external wall of the second chamber is transparent and includes volumetric indication markings.

21. The urometer of claim 18, wherein the first chamber further comprises a drainage bag in the first chamber having a top wall and bottom wall defining an inner space characterized by a width or a horizontal depth greater than a height.

22. A method of collecting a biological fluid from an animal, the method comprising:
catheterizing the animal with a catheter;
providing a collection bag comprising an inner space defined between a top wall and a bottom wall, wherein the inner space is characterized by a width or a horizontal depth greater than a height; and wherein the drainage tube is in fluid contact with the bag inner space through an inlet port located within the top wall; and,
functionally connecting a drainage tube between the catheter and the collection bag and draining the biological fluid from the catheter into the bag inner space through the drainage tube.

23. The method of claim 22, further comprising mounting or resting the bag at a location below a location where the catheter is mounted into the animal.

24. The method of claim 22, further comprising placing the bag on a horizontal surface with the bottom wall resting on the horizontal surface.

25. The method of claim 24, wherein the horizontal surface is a floor of a room.

26. The method of claim 22, wherein the inlet port is closer to a center of the top wall than to a peripheral edge of the top wall.

27. A drainage collection system comprising:
a collection receptacle with an internal space and having a low aspect ratio; and,
an inlet port located above the internal space.

28. The system of claim 27, wherein the collection receptacle not substantially flexible.

29. The system of claim 27, wherein the aspect ratio is 0.3 or less.

30. The device of claim 27, further comprising a collar mounted to the device above the inlet port, which collar has a conical inner surface expanding upwards.
